(19) United States
(12) Patent Application Publication
(10) Pub. No.: US 2008/0021350 A1
(43) Pub. Date: Jan. 24, 2008

(54) Cervix Measurment Device with Disposable Tip

(76) Inventors: Samuel J. Bechtle, San Clemente, CA (US); Vincent J. Bianco, San Clemente, CA (US)

Correspondence Address: BLAKELY SOKOLOFF TAYLOR & ZAFMAN, 1279 OAKMEAD PARKWAY, SUNNYVALE, CA, 94085-4040, US

(21) Appl. No.: 11/457,427

(22) Filed: Jul. 13, 2006

Related U.S. Application Data
(60) Provisional application No. 60/699,563, filed on Jul. 15, 2005.

(51) Int. Cl. A61B 5/103

(52) U.S. Cl. 600/588

(57) ABSTRACT

The present invention provides several devices and methods for determining dimensions of female reproductive organs. In one embodiment, a device for determining a dimension of a female reproductive organ includes a hollow member having a body, a distal end, and a portion extending substantially perpendicular to the body, a measurement member having a body, a proximal end, a closed distal end, and a measurement scale along a length of the body, and a probe member having a body, a distal end, a proximal end, and a light-emitting element. The probe member is configured to be inserted into the body of the measurement member, and the measurement member is configured to be inserted into the body of the hollow member. The measurement member and the hollow member may be disposable and may be discarded after use.

### FIELD OF THE INVENTION

The present invention relates to medical devices and methods of using such devices. More particularly, the invention relates to instruments and methods to measure the length of the cervix in the fornix vaginae and the dilation of the cervix uteri.

### BACKGROUND

Preterm labor, or labor before 37 weeks gestation, has been reported in 7 to 10 percent of all births but accounts for more than 85 percent of all perinatal complications and death. Rush et al., BMJ 2:965-8 (1976) and Villar et al., Res. Clin. Forums 16:9-33 (1994), which are both incorporated herein by reference. An inverse relationship between cervical length in the fornix vaginae and the risk of preterm labor has also been observed. Anderson et al., Am. J. Obstet. Gynecol. 163:859 (1990); Iams et al., N. Eng. J. Med. 334:567-72 (1996) and Heath et al., and Ultrasound Obstet. Gynecol. 12:312-7 (1998), which all are incorporated herein by reference. Accordingly, many physicians find it useful to examine the cervix in the fornix vaginae as part of normal prenatal care in order to assess risk of preterm labor.

It has long been known that the cervix normally undergoes a series of physical and biochemical changes during the course of pregnancy, which enhance the ease and safety of the birthing process for the mother and baby. For example, in the early stages of labor the tissues of the cervical canal soften and become more pliable, the cervix shortens (effaces), and the circumference of the proximal end of the cervical canal begins to increase at the internal os. As labor progresses, growth of the cervical diameter propagates to the distal end of the cervical canal, toward the external os. In the final stages of labor, the external os dilates allowing for the unobstructed passage of the fetus.

In addition to the physical and biochemical changes associated with normal labor, genetic or environmental factors, such as medical illness or infection, stress, malnutrition, chronic deprivation and certain chemicals or drugs can cause changes in the cervix. For example, it is well known that the in utero exposure of some women to diethylstilbestrol (DES) results in cervical abnormalities and in some cases gross anatomical changes, which leads to an incompetent cervix where the cervix matures, softens and painlessly dilates without apparent uterine contractions. An incompetent cervix can also occur where there is a history of cervical injury, as in a previous traumatic delivery, or as a result of induced abortion of the cervix is forcibly dilated to large diameters. Details of the incompetent cervix are discussed in Sonek, et al., Preterm Birth, Causes, Prevention and Management, Second Edition, McGraw-Hill, Inc., (1993), Chapter 5, which is incorporated by reference herein.

Cervical incompetence is a well-recognized clinical problem. Several investigators have reported evidence of increased cervical os diameter as being consistent with cervical incompetence (see Brook et al., J. Obstet. Gynecol. 88:640 (1981); Michaels et al., Am. J. Obstet. Gynecol. 154:537 (1986); Sarti et al., Radiology 130:417 (1979); and Vaalamo et al., Acta Obstet. Gynecol. Scan 62:19 (1983), all of which are incorporated by reference herein). Internal os diameters ranging between 15 mm to 23 mm have been observed in connection with an incompetent cervix. Accordingly, a critical assessment in the diagnosis of an incompetent cervix involves measurement of the internal cervical os diameter.

There are also devices and methods to measure the diameter of the external cervical os. For example, cervical diameter can be manually estimated by a practitioner's use of his or her digits. Although an individual practitioner can achieve acceptable repeatability using this method, there is significant variation between practitioners due to the subjective nature of the procedure. To address these concerns, various monitoring and measuring devices and methods have been developed. For example, an instrument for measuring dilation of the cervix uteri is described in U.S. Pat. No. 5,658,295. However, this device is somewhat large, leading to a risk of injury to the fundus of the vagina or cervical os. Additionally, it is not disposable and requires repeated sterilization. Another device for measuring cervical diameter is described, for example, in U.S. Pat. No. 6,039,701. In one version, the device described therein has a loop element that is secured to the cervix. The loop expands or contracts with the cervix and a gauge is coupled to the loop for measuring changes in the loop dimension. Such changes can then be detected by electronic means. Accordingly, this device is rather complex and expensive to manufacture.

Even if a woman is found to have an apparently normal internal cervical os diameter, there may nonetheless be a risk for preterm labor and delivery. Currently, risk assessment for preterm delivery remains difficult, particularly among women with no history of preterm birth. However, the findings that preterm delivery is more common among women with premature cervical shortening or effacement suggest that measuring the length of the cervix would be predictive for preterm labor.

Currently, a physician has at least two options to measure the length of the cervix in the fornix vaginae. One such method involves serial digital examination of the cervix by estimating the length from the external cervical os to the cervical-uterine junction, as palpated through the vaginal fornix. Although this is useful for general qualitative analysis, it does not afford an easy nor accurate measurement of the length of the cervix from the external cervical os to the cervical-uterine junction (also described herein as the length of the cervix extending into the vagina) and, therefore, does not provide an accurate assessment of the risk of preterm labor. Despite the use of gloves, vaginal exams always carry with them the risk of transmitting infectious agents, especially to the fetal membranes, the lining and/or muscle of the uterus, or the fetus itself

Another method involves real-time sonographic evaluation of the cervix. This method provides relatively quick and accurate cervical dimensions. However, it requires expensive equipment, highly skilled operators, as well as skill in interpretation of results, which are all subject to human error. Also, due to the expense of the procedure many women, especially those without proper health insurance, cannot afford to have a sonographic test performed.

It would be beneficial if there were an instrument a practitioner could use to measure the cervix quickly and accurately, and with little material expense. Although there are several instruments available for determining various dimensions of the uterus, there is no suitable instrument for measuring the length of the cervix in the fornix vaginae. For example, U.S. Pat. No. 4,016,867 describes a uterine caliper and depth gauge for taking a variety of uterine measurements, which although useful for fitting an intrauterine contraceptive device, is not capable of measuring the length of the cervix in the fornix vaginae due to interference by the caliper's wings. In fact, similar devices described in U.S. Pat. Nos. 4,224,951; 4,489,732; 4,685,747; and 5,658,295 suffer from similar problems due to their use of expandable wings or divergable probe tips. These devices are also relatively sophisticated, making them expensive to manufacture and purchase. U.S. Pat. No. 3,630,190 describes a flexible intrauterine probe, which is particularly adapted to measuring the distance between the cervical os and the fundus of the uterus. The stem portion of the device has a plurality of annular ridges spaced apart from each other by a predetermined distance, preferably not more than one-half inch apart. This device is not, however, adapted for accurately measuring the length of the cervix in the fornix vaginae because of the lack of an appropriate measuring scale and a stop for automatically recording the measurement.

Accordingly, there is currently no commercially available, quick, and inexpensive as well as accurate device to assess the risk of preterm labor by measuring the length of the cervix in the fornix vaginae. Therefore, many women at risk for preterm labor may be unaware of the risk to their pregnancy and their unborn child. If such a device were available, many more women would be better informed about the course of their pregnancy and would then be able to make better choices about becoming pregnant at all, or about managing their pregnancy to reduce the risk of preterm labor and injury to the unborn child.

Thus, there exists a need for a simple and inexpensive device that can be used to determine the length of the cervix in the fornix vaginae and, thus, predict the risk of preterm labor, as well as other conditions. There is also a need for such a device that can measure the dilation of the cervix uteri, to provide an overall assessment of the cervix and to determine the particular stage of labor. Ideally, the device should be adapted for use by a physician or obstetrician or even a trained nurse in the doctor's office or clinic. Preferably, the device should be disposable or capable of being sterilized. In addition, it is desirable that device record the measurement automatically. The present invention satisfies these needs and provides related advantages as well.

### SUMMARY OF THE INVENTION

The present invention provides devices and methods for determining a dimension of a female reproductive organ.

In one aspect of the present invention, a device for determining a dimension of an organ may include a hollow member with a distal end, a proximal end, and a lumen therebetween, a measurement member insertable into the hollow member and having a distal portion, a proximal portion, a measurement scale disposed along the proximal portion, and a lumen therebetween, and a probe member with a distal portion and a proximal portion, wherein the probe member may be inserted into the lumen of the measurement member. Additionally, the hollow member may include a flange having a body offset substantially perpendicular to the hollow member, wherein the flange is attached near the distal end of the hollow member. The probe member may include a handle containing a replaceable battery, a body configured to be inserted into the lumen of the measurement member, a light-emitting element disposed near the distal end of the body, and a clamping cap to secure the handle to the body of the probe member and the measurement member.

Other objects and features of the present invention will become apparent from consideration of the following description taken in conjunction with the accompanying drawings.

### DESCRIPTION OF THE DRAWINGS

FIG. 1 illustrates a device of the present invention having a measurement member, a probe member, which may be lighted, and a hollow member, with the probe member inserted into the measurement member and the measurement member inserted into the hollow member.

FIG. 2 illustrates a device of the present invention having the probe, which may be lighted, member withdrawn from the measurement member and hollow member.

### DETAILED DESCRIPTION

The present invention provides various devices and methods for determining dimensions of female reproductive organs. For example, the device is particularly adapted for determining the length of the cervix in the fornix vaginae, which, as described above, is related to the risk of preterm labor in an individual. The device is also suited for determining the dilation of the cervix uteri, for predicting the risk of preterm labor or the particular stage of delivery. It is, however, contemplated herein, and also understood by those skilled in the art, that the invention can be used not only for determining various dimensions of female reproductive organs. For example, the invention is usable for determining the dimension of any body cavity or passageway where such a device would be insertable, such as a vagina, uterus, mouth, throat, nasal cavity, ear channel, rectum, and also to any cavity created and opened by surgery, for example, during chest, abdominal or brain surgery. It is also contemplated that the device records the various measurements automatically, where the only input required by the practitioner is the proper insertion of the device into the body cavity or passageway. This is accomplished by the use of the flange to stop progression of the hollow member of the device while still allowing the measurement member to be advanced within the body.

As used herein the term “distal” refers to the end or portion of a device of the present invention, or a component thereof, which is adapted to be inserted first into a body cavity or passageway. As such, it will be that part of a device of the present invention furthest from the user while the device is inserted and progressed within the body cavity. Conversely, as used herein, the term “proximal” refers to the end or portion of the device nearest the user while the device is being inserted and progressed within the body cavity.

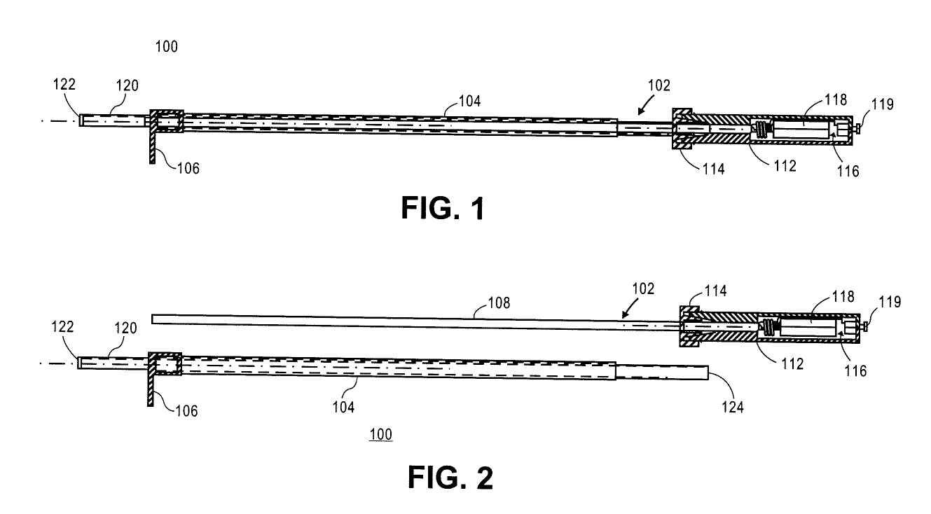

Turning to FIGS. 1 and 2, a measuring device 100 of the present invention is illustrated. Measuring device 100 includes an elongated hollow member 104 having a distal region with a distal end, a proximal region with a proximal end, and a lumen extending between the distal and proximal ends. The hollow member 104 may include a flange 106 on the distal region. The flange 106 is shaped for non-abrasive contact with tissue. The flange 106 is preferably flat and may be spherically or conically shaped. Alternatively, however, the flange 106 may be any other non-abrasive shape to reduce irritation and scraping of the cervical canal, fundus of the vagina or perforation of the fundus of the uterus. The main body of flange 106 is also preferably offset from the longitudinal axis of the hollow member 104. Preferably, the flange 106 is secured to the distal end of the hollow member 104 using a suitable attachment means, such as, e.g., an adhesive or press fit. Alternatively, the flange 106 may be formed as an integral component of the hollow member 104. In another embodiment of the device 100, the flange 106 further includes a plurality of measurement marks that are, for example, usable for measuring the dilation of the cervix or external uterine opening. Here, the flange 106 is preferably manufactured from a substantially transparent or translucent material, such as plastic, so that the user is able to observe the flange 106 while the flange 106 is placed within the body. After the flange 106 contacts the external uterine opening, the user is able to measure the dilation of the cervix by comparing the size of the external uterine opening with the measurement marks on the flange 106. The measurement marks may be metric, such as, e.g., incremental marks of millimeters, centimeters, or a combination thereof, or in English scale, such as, e.g., inches.

The measuring device 100 also includes a measurement member 120 having a distal end, a proximal end, and a lumen therebetween. The measurement member 120 preferably has a closed distal end 122 and an open proximal end 124. The measurement member 120 is adapted to be inserted into the lumen of the hollow member 104. In one embodiment, the hollow member 104 and the measurement member 120 are designed to be disposed after use of the measuring device 100, and are therefore formed from a suitable disposable material such as, e.g., plastic or a similar material.

In one embodiment of the measuring device 100, a measurement scale may be disposed along the proximal portion of the measurement member 120. As used herein, the “measurement scale” refers to any number of a series of visual markings on the measurement member 120 at or near the proximal end, which relate a measurement or distance. In a particularly preferred embodiment, the measuring scale includes a plurality of millimeter (mm) incremental markings and a plurality of centimeter (cm) incremental markings. Furthermore, the measurement scale may include relatively larger markings at 1, 2, 3, 4, and 5 cm in addition to a plurality of millimeter incremental markings between the centimeter markings. Additionally, a critical mark may be present at approximately 2.5 cm. In one embodiment, the critical mark is presented in a different color, such as, e.g., a red color, relative to the other incremental markings of the measurement scale. The critical mark is used to quickly notify a user of device 100 that a particular cervix length represents a greater risk of preterm delivery relative to longer cervix lengths. In another embodiment of the device 100, the measurement scale is coded into a plurality of regions. For example, in one implementation of this embodiment, the incremental markings less than 2 cm are coded in a first color, such as, e.g., red, the incremental markings from 2 to 3 cm are coded in a second color, such as, e.g., yellow, and the incremental markings from 3 to 5 cm are coded in a third color, such as, e.g., green. In this embodiment, the measurement scale is color-coded into three regions that each visually represents the relative risks of preterm delivery for a cervix length falling within the respective region. In the above described example, for instance, the red zone indicates a shorter cervix, and therefore a higher risk of preterm delivery, than the yellow zone, which indicates a cervix length that reflects a higher risk of preterm delivery than the green zone. The measurement scale is capable of being coded into regions based upon other distinguishing marks also, such as, e.g., a first region having a first type of marking for the measurement increments, a second region having a second type of marking for the measurement increments, and a third region having a third type of marking for the measurement increments. In other embodiments of device 100, the incremental markings are in English measurements, such as inches and increments thereof, rather than the metric increments previously discussed.

A probe member 102 is provided and is designed to be inserted into the lumen of the measurement member 120. The probe member 102 includes an elongate body 108 that is capable of being progressed within the measurement member 120 both proximally and distally. In one embodiment, the elongate body 108 is formed from a metallic material. In another embodiment, the elongate body 108 is formed from a plastic material. In the illustrated embodiment of probe member 102, attached to the proximal end of the elongate body 108 is a handle 112. A clamping cap 114 may be provided to secure the handle 112 to the elongate body 108. The clamping cap 114 on handle may be designed to restrict rotation of the measurement m ember 120 relative to handle 112. The handle 112 may be molded from the same material as the elongate body 108. The handle 112 preferably includes a cavity 116 configured to accommodate a battery 118. The probe member 102 further incorporates a light-emitting element within the elongate body 108. The light-emitting element is a component capable of generating light that is directed substantially in a distal direction when the device 100 is placed within the body. The light-emitting element may be any suitable light source, such as, e.g., a light-emitting diode, a laser, an incandescent light bulb, a fluorescent substance, or the like. In another embodiment, the light-emitting element is an array of individual light-emitting components rather than a single light-emitting component. When an array of individual light-emitting components are used instead of a single light-emitting component, the light-emitting element is capable of continually emitting light in the event one of the array of light-emitting components fails during the operation of device 100. In one embodiment, the light-emitting element is disposed at or near the distal end of the elongate body 108. Electrical leads couple the light-emitting element to the battery 118, and an activation component 119 is provided on the handle 112 that when operated completes an electrical circuit between the battery 118 and the light-emitting element.

To allow for light to pass therethrough, measurement member 120 is preferably manufactured from a substantially transparent or translucent material such as plastic. In one embodiment, the entire measurement member 120 is constructed from a substantially transparent or translucent material. In another embodiment, the distal portion of the measurement member 120 is constructed from a substantially transparent or translucent material while the remaining portion of the member 120 may be constructed from a substantially opaque material such as a metallic substance.

In an embodiment of the invention the measurement device 100 may be configured to interlock to reduce the amount of rotation of the measurement member 120 within the hollow member 104. Here, the device 100 incorporates a hollow member 104 and a measurement member 120 that allows for the hollow member 104 to travel longitudinally over the measurement member 120 when in an unlocked position. The interlocking features of this embodiment allow for a user to fix the position of the measurement member 120 within the hollow member 104 when desired, such as, e.g., after a measurement of a body part has been taken, by rotating the handle 112 and thus rotating the measurement member 120 within the hollow sleeve 104.

With this embodiment the measurement member 120 and the hollow member 104 are not circular in shape. Instead, hollow member 104 and measurement member 120 may all be rectangular, octagonal, square, or another shape having at least one angle such that rotation of the measurement member 120 within the hollow member 104, is substantially reduced and prevented by the angles of the measurement member 120 and hollow member 104. For example, the measurement member 120 and hollow member 104 may all have oval cross-sections. Here, the measurement member 120 is still capable of being placed within the hollow member 104, and being advanced distally and proximally when the cross-sections are in a substantially parallel relationship. Due to the relative cross-sectional shapes of the members, however, the ability to rotate the measurement member 120 while traveling within the hollow member 104 may be restricted by rotating the measurement member 120 until the sides of the oval cross-section engage the inner walls of the hollow member 104 such that the oval cross-sections are no longer in a parallel relationship.

Another interlocking embodiment of the device 100 is provided. With this embodiment, the measurement member 120 may include a flat face that extends along a portion of the length of the member. Additionally, the hollow member 104 may include a ridge or protrusion along the inner wall of the member. When it is desired to advance the measurement member 120 distally or proximally within the hollow member 104, the flat face on the length of the measurement member 120 is oriented towards the ridge or protrusion on the inner wall of the hollow member 104, thereby allowing distal or proximal movement of the measurement member 120 within the hollow member 104. Then, when it is desired to lock the position of the measurement member 120 relative to the hollow member 104, the measurement member 120 is rotated such that the ridge or protrusion on the inner wall of the hollow member 104 contacts an outer edge of the flat face on the length of the measurement member 120.

With regard to materials of manufacture, the devices of the present invention are capable of being formed from either metallic materials or plastic materials. In one embodiment, the devices are manufactured from a metal, such as, e.g., brass, stainless steel, aluminum, or the like, using techniques known in the art. When formed from a metallic material, the devices may be sterilized using an appropriate means, including, e.g., chemical sterilization, thermal sterilization, radiation, and the like. In another embodiment, the devices of the present invention are formed from a plastic material. With these embodiments, the devices may be extruded from plastic using techniques known in the art.

In one method of use, the measurement device 100 may be used to measure the length of a cervix. The elongate body 108 of the probe member 102 is placed within the lumen of the measurement member 120, and the measurement member 120 is placed within the lumen of the hollow member 104. The measurement member 120 is preferably arranged such that not a significant portion of the measurement member 120 extends distally beyond the hollow member 104 prior to insertion of the device 100 within a patient's body. The measurement device 100 is then inserted into the vagina and advanced until flange 106 is placed into contact with the end of the cervix at the external uterine opening. At this point, further forward progress of the hollow member 104 within the cervical canal or further within the body is substantially prevented as a result of the contact between flange 106 and the end of the cervix at the external uterine opening. Since flange 106 is preferably offset from the longitudinal axis of the hollow member 104, and therefore from the longitudinal axis of the device 100, in one manner of operation optimal for measuring the length of the cervix, flange 106 is placed both in contact with the end of the cervix and also covering the external uterine opening. As a result, device 100 is oriented so that measurement member 120 and the elongate body 108 of the probe member 102 are still able to be progressed within the fornix, rather than being advanced through the uterus, since the body of flange 106 is, with this method, covering the external uterine opening. Subsequently, the measurement member 120 is advanced distally beyond the hollow member 104. To assist with maneuvering the measurement member 120, the activation component 119 may be operated to turn on the light-emitting element. The measurement member 120 (with the probe member 102 inside) is advanced until the closed distal end 122 of the measurement member 120 contacts a wall of the body, such as, e.g., the anterior fornix. After the desired measurements are taken, the measurement device 100 may be removed from the body and the hollow member 104 and measurement member 120 disposed. The length of the cervix is determined by observing the position of the proximal end of the hollow member 104 along the measurement scale on the measurement member 120. This observation may be performed either when the device 100 is still in the patient's body or after removal of the device 100 from the patient's body.

If light is not required to maneuver the device 100 within the patient's body, the measurement member 120 may be advanced within the body without use of the probe member 102 and handle 112. The operation of this embodiment is otherwise the same as in the above description of the use of the device 100.

Although the invention has been described with reference to the examples provided above, it should be understood that various modifications can be made without departing from the spirit of the invention. Accordingly, the invention is limited only by the claims.

What is claimed is:

1. A device for determining a dimension of an organ comprising:
a hollow member having a body, a distal end, and a portion extending substantially perpendicular to the body,
a measurement member having a body, a proximal end, a closed distal end, and a measurement scale along a length of the body, and
a probe member having a body, a distal end, a proximal end, and a light-emitting element,
wherein the probe member is configured to be inserted into the body of the measurement member, and the measurement member is configured to be inserted into the body of the hollow member.

2. The device of claim 1, wherein the measurement member interlocks with the hollow member to secure a position of the measurement member within the hollow member.

3. The device of claim 1, wherein the portion of the hollow member extending substantially perpendicular to the body is disposed on the distal end of the hollow member.

4. The device of claim 1, wherein the portion of the hollow member extending substantially perpendicular to the body comprises a shape chosen from the group consisting of a tear-drop shape and a circular shape.

5. The device of claim 1, wherein the probe member comprises a handle disposed on the proximal end.

6. The device of claim 5, wherein the handle is coupled to the proximal end of the measurement member with a clamping cap.

7. The device of claim 5, wherein the handle comprises a cavity and a battery disposed in the cavity.

8. The device of claim 6, wherein the battery is connected to the light-emitting element.

9. The device of claim 1, wherein the body of the probe member is formed from a metal.

10. The device of claim 1, wherein the body of the probe member is formed from plastic.

11. The device of claim 1, wherein the hollow member and the measurement member are formed from plastic.

12. A method for measuring a length of a cervix, comprising:
inserting a device into a vagina, the device comprising a hollow member having a body and a distal end having a flange, a measurement member having a body, a proximal end, a closed distal end, and a measurement scale along a length of the body, and a probe member having a body, a distal end, a proximal end, and a light-emitting element, wherein the probe member is configured to be inserted into the body of the measurement member, and the measurement member is configured to be inserted into the body of the hollow member,
advancing the device distally within the vagina until the flange contacts a cervix at an external uterine opening,
advancing the measurement member until the distal end of the measurement member contacts a cervical-uterine junction at a fornix vaginae, and
observing a position of the proximal end of the hollow member along the measurement scale to determine the length of the cervix in the fornix vaginae.

13. The method of claim 12, comprising illuminating the vagina using the light-emitting element.

14. The method of claim 12, comprising locking the measurement member relative to the hollow member prior to observing a position of the proximal end of the hollow member along the measurement scale.

15. The method of claim 12, comprising prior to inserting the device into the vagina positioning the measurement member within the hollow member such that the distal end of the measurement member does not extend distally beyond the distal end of the hollow member.
